(19) United States
(12) Patent Application Publication
(10) Pub. No.: US 2025/0134380 A1
(43) Pub. Date: May 1, 2025

(54) DETECTION AND LOCALIZATION OF DISEASE UTILIZING FLUORESCENCE SPECTRA OF EXOGENOUS FLUOROPHORES

(71) Applicants: PREVIEW MEDICAL INC., Longmont, CO (US); THE REGENTS OF THE UNIVERSITY OF COLORADO, A BODY CORPORATE, Denver, CO (US)

(72) Inventors: Sharon Lyn LAKE, Longmont, CO (US); Priya Niranjan WERAHERA, Aurora, CO (US)

(73) Assignees: PREVIEW MEDICAL INC., Longmont, CO (US); THE REGENTS OF THE UNIVERSITY OF COLORADO, A BODY CORPORATE, Denver, CO (US)

(21) Appl. No.: 18/835,845

(22) PCT Filed: Feb. 3, 2023
(86) PCT No.: PCT/US2023/061985

§ 371 Date: Aug. 5, 2024

Related U.S. Application Data
(60) Provisional application No. 63/307,816, filed on Feb. 8, 2022.

(51) Int. Cl. A61B 5/00

(52) U.S. Cl. CPC A61B 5/0071; A61B 5/0082; A61B 5/4848

(57) ABSTRACT

The present discloses systems and methods for tissue analysis, biopsy, and treatment utilizing at least exogenous fluorophores and near infrared light. A method is disclosed for optically determining the presence of abnormal cells, such as cancer, in tissue. The method includes applying one or more exogenous fluorophores into tissue in an animal body, wherein the exogenous fluorophores include a targeting moiety formulated to bond with a selected site on abnormal cells. The method includes illuminating the one or more exogenous fluorophores in the tissue with near infrared light from an elongated optical probe to generate fluorescent light from exogenous fluorophores present in the tissue. The method includes receiving fluorescent light generated from the one or more exogenous fluorophores in the tissue. The method includes determining if abnormal cells are present in the tissue based on the fluorescent light generated from the one or more exogenous fluorophores in the tissue.

### CROSS REFERENCED TO RELATED APPLICATIONS

This application claims the benefit of priority to U.S. Provisional Patent Application No. 63/307,816, filed Feb. 8, 2022, entitled “DETECTION AND LOCALIZATION OF DISEASE UTILIZING FLUORESCENCE SPECTRA OF EXOGENOUS FLUOROPHORES” the disclosure of which is hereby incorporated in its entirety and for all purposes.

### BACKGROUND

Cancer is a highly destructive disease, resulting in large numbers of deaths every year. Current treatments of cancer, whether be surgical, chemical, or radiological, have a number of complications that can greatly diminish quality of life of patients. A recognized way to avoid these complications centers around early detection of malignant cells, or of cell properties that are precursors to malignancy. Treatment options are far less invasive and damaging when these conditions are detected early and with disease localized to the organ and treatments are directed to either specific regions or lesions as opposed to whole organ.

Currently, precancerous molecular or chromosomal abnormalities that may precede cancerous cells are largely undetectable. In addition, sampling errors can occur, where the location analyzed appears normal, however the cancer is present and located elsewhere, leading to false-negative biopsies. Further, a positive biopsy at one site may not give a full picture of the extent of the disease if the cancer is multifocal or has extended to other locations, e.g., other areas of the organ or adjacent structures, such as connected tissue and lymph nodes. Other than by histopathological examination, currently there are no standard techniques for determining morphology of genetically abnormal neoplastic tissue from benign tissue at resection margins in operating rooms. Failure to completely remove such abnormalities may lead to recurrence of the disease.

Opportunities to intervene may be enhanced if accurate detection of the pre-disease/disease state can be achieved at the biochemical, structural, or pathological/physiological levels. However, many cancer diagnoses require histopathological confirmation of the disease by examining biopsy tissues. Biopsy or tissue collection tools may be difficult to use depending on the location of the potential cancerous lesion. This leaves many cancers undetected until an advanced stage, reducing survival, and requiring more aggressive treatment options. Accordingly, medical professionals continue to search for options for better screening of tissues.

### SUMMARY

Embodiments relate to systems and methods for optical tissue analysis, biopsy, and/or treatment utilizing at least exogenous fluorophores and at least near infrared (NIR) light.

In an embodiment, a method for optically determining the presence of abnormal cells in tissue is disclosed. The method includes applying one or more exogenous fluorophores into tissue in an animal body, wherein the exogenous fluorophores include a targeting moiety formulated to either bond or increased uptake within a selected site on abnormal cells. The method includes illuminating the one or more exogenous fluorophores in the tissue with near infrared light from an elongated optical probe to generate fluorescent light from exogenous fluorophores present in the tissue. The method includes receiving fluorescent light generated from the one or more exogenous fluorophores in the tissue. The method includes determining if abnormal cells are present in the tissue based on the fluorescent light generated from the one or more exogenous fluorophores in the tissue.

The method may include illuminating one or more endogenous fluorophores in the tissue with visible light, ultra-violet light, or near infrared light from the elongated optical probe to generate fluorescent light from the endogenous fluorophores present in the tissue. The method includes receiving fluorescent light generated from the one or more endogenous fluorophores in the tissue. The method includes identifying if healthy cells are present in the tissue based on the fluorescent light generated from the one or more endogenous fluorophores in the tissue. The method includes determining a relative location of the healthy cells and the abnormal cells. The method includes locating margins between healthy cells and abnormal cells based on position data of the fluorescent light generated from the one or more exogenous fluorophores and one or more endogenous fluorophores in the tissue. The method includes applying a treatment to the abnormal cells. The method includes differential analysis to determine efficacy of treatment based on the location of the healthy cells and the abnormal cells before and after a treatment. The method includes building a three-dimensional map of relative health of tissue by moving the optical probe to a new location longitudinally spaced from a prior location by a distance and repeating the applying, illuminating, receiving and determining steps with UV, visible, and/or NIR light and repeating the illuminating, receiving, identifying, and locating steps with visible light to determine a location of healthy cells and abnormal cells.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the invention, wherein identical reference numerals refer to identical or similar elements or features in different views or embodiments shown in the drawings.

FIG. 1 is a block diagram of an optical probe system, according to at least some embodiments.

FIG. 2A is an isometric view of an in vivo optical probe, according to an embodiment.

FIG. 2B is a left end elevation view of the optical probe of FIG. 2A, according to an embodiment.

FIGS. 3A-3B are isometric views of a minimally invasive automatic optical probe, according to an embodiment.

FIG. 4 is a block diagram of the computer, according to an embodiment.

FIG. 5 is a flow diagram of a method for optically determining the presence of abnormal cells in tissue, according to an embodiment.

### DETAILED DESCRIPTION

Embodiments relate to optical systems and methods of using the same to identify health of a tissue, such as determine the presence or absence of abnormal cells (e.g., suspicious or cancer cells, necrotic cells, and other non-normal cells) or biomarkers of the same in the tissue based on fluorescent light emitted from exogenous fluorophores (e.g., organic/inorganic compounds, quantum dots, nanoparticles, etc.) bound thereto when illuminated by NIR light. The systems and methods disclosed can be used as is or adopted at one or more stages for clinical management of disease, such as, for example: to diagnose (e.g., indicating cancer vs benign), aggressiveness (e.g., histopathological grade of cancer) or localize (e.g. stage of cancer) disease, to guide interventions, or to monitor treatment response and and/or disease progression in case of recurrence or the like. The systems and methods disclosed herein can use endogenous fluorophores (e.g., tryptophan, collagen, nicotinamide adenine dinucleotide with hydrogen (NADH), flavin adenine dinucleotide (FAD), etc.), exogenous fluorophores, or a combination thereof to determine the presence of abnormal cells in selected tissue, margins between abnormal cells and normal or health cells, and progress of treatments based on differential analysis of the tissue. As used herein, the term abnormal is meant to encompass all types of unhealthy or different cells or tissue, including, but not limited to, cancer cells, suspicious or diseased cells, necrotic cells, treated cells, or the like. The term healthy or normal cells are meant to encompass cells and tissue that are not abnormal, e.g., are not cancerous, diseased, and so on. The various methods and systems can detect the difference between abnormal and normal cells, such as to identify location of abnormal cells, boundaries between abnormal and normal cells, and so on.

Light does not penetrate deep into tissue in the UV and part of the visible range of 250-650 nm, but the remainder of the visible range and NIR wavelengths ranging from 650 to 1350 nm has its maximum depth of penetration up to several centimeters into tissue. Within this NIR wavelength range also known as “therapeutic window,” photon absorption by hemoglobin and water diminishes, leading to deeper tissue penetration of light than UV or visible light. Furthermore, tissue auto-fluorescence is low in the NIR spectra, which allows a high signal to noise ratio for optical spectra. Thus, endogenous fluorophores provide little to no interference with exogenous fluorophores in the NIR spectrum. There are several types of organic and inorganic exogenous fluorophores that work specifically in the NIR spectrum to capitalize on above advantages to diagnose and treat diseases. The exogenous fluorophores can be modified with targeting moieties which target specific organs or abnormal cells such as cancer cells, abnormal benign cells, atypical cells, etc., or biomarkers associated with the foregoing to bind with such structures to provide fluorescent light indicating the presence of said structures. Such exogenous fluorophores are utilized with the systems and methods herein to identify or categorize tissue such as to identify abnormal or normal tissue (e.g., identify presence of cancer cells, suspicious areas, treatment success, necrotic cells, etc.) based on the detectability by an optical probe of fluorescent light characteristics of the exogenous fluorophores. For example, the exogenous fluorophores may be preferentially bound and in greater concentration on certain types of tissue (e.g., suspicious cells or biomarkers associated therewith) than on healthy tissue.

Exogenous fluorophores synthesized to absorb NIR light provide a stronger fluorescence spectra resulting in increased sensitivity and detection of unhealthy cells (e.g., suspicious cells)—in vivo and in vitro—compared to endogenous fluorophores that typically interact with light in the visible and UV spectra. By attaching the exogenous fluorophores to selected cells, tissue, and/or biomarkers, the systems and methods disclosed can easily identify the presence or absence of the selected cells, tissue, or biomarkers using NIR light in the range corresponding to the exogenous fluorophores. Such exogenous fluorophores can be utilized to identify abnormal cells at the surface of the tissue and below the surface of the tissue because NIR light can penetrate at least several centimeters into tissues. The lack of interference between light fluoresced from endogenous fluorophores and exogenous fluorophores (composed to interact with NIR light) provides clear distinctions between the various types of fluoresced light in the tissue. Such distinctions allow for relatively interference-free interpretation of the spectra to determine if the fluoresced light indicates a specific tissue has abnormal cells (e.g., malignant cancer cells), or only normal cells.

Conventionally, exogenous fluorophores composed to emit in the NIR range have not been used for in vivo optical probing and/or biopsies because they can be toxic, do not bind well to tissue or biomarkers, or are relatively rapidly cleared from the tissue by natural biological processes (e.g., renal clearance). However, the systems and methods disclosed here provide exogenous fluorophores that have targeting moieties composed to bind with selected tissue (e.g., organs), abnormal cells, or biomarkers of the same to provide targeted examination of tissue. The exogenous fluorophores also may include a coating helping to prevent toxic side effects if toxic materials are present in the fluorophores. Accordingly, the systems and methods disclosed leverage NIR light with exogenous fluorophores to identify abnormal tissue, cancerous, tissue or healthy tissue in vivo or in vitro.

In one example, an optical probe including optical fibers and one or more processing elements that can emit and detect light in the NIR spectra is disclosed. Such optical probes can be used to evaluate tissue characteristics in real time and in vivo to provide a mapping and tissue characterization. In some embodiments, the optical fiber may include additional fibers and/or processing elements that emit and detect light outside of the NIR spectra, allowing the optical probe to be used to detect tissue characteristics without the addition of exogenous fluorophores, as well as tissue with the exogenous fluorophores. In other embodiments, the optical fibers outside of the NIR spectra may be used separately or in addition to the NIR optical probe. The configuration of the optical probe and particularly the light sensing and analyzing elements may be varied as desired depending on hardware configurations and desired uses. For example, the optical probe may include a first set of optical fibers configured to detect NIR spectra and a second set of optical fibers configured to detect non-NIR spectra, where the probe may include separate processing elements to analyze the signals from the first and second sets of optical fibers, respectively, or may include a single processing element configured to analyze signals from both sets of optical fibers. In this manner, the optical fibers may be integrated into the same probe and used at the same time, or may be separately activated, or may be used as two discrete devices in the same location.

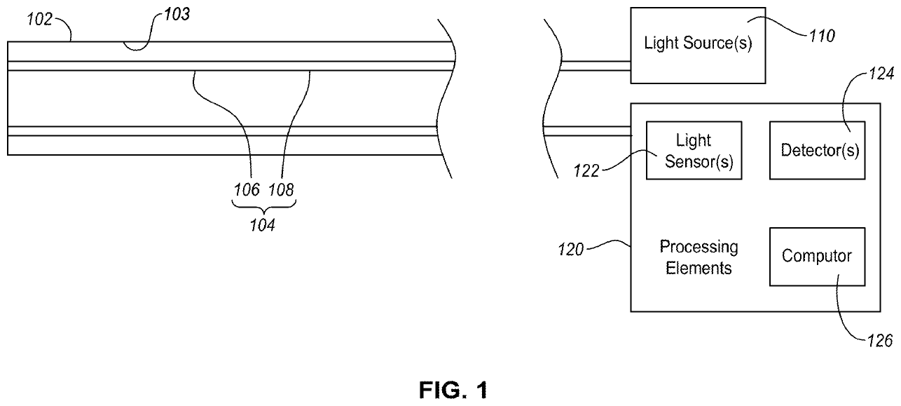

FIG. 1 is a block diagram of an optical probe system 100, according to at least some embodiments. The system 100 can be used for optical spectroscopy and optional physical biopsies of tissues. The system 100 includes the optical probe 102, a plurality of optical fibers 104 including at least one transmitting fiber 106 and at least one receiving fiber 108, at least one light source 110 constructed to emit light in at least the NIR spectrum, and one or more processing elements 120 configured to analyze light received by the optical fibers 104 and determine whether a tissue is healthy based on the received light (e.g., determine the presence of abnormal cells in the tissue). The system 100 is used to irradiate tissue having exogenous fluorophores administered therewith at least NIR light, receive fluorescent light from the exogenous fluorophores, and determine whether abnormal cells or normal cells are present in the tissue.

The optical probe 102 is an optical probe having the plurality of optical fibers 104 therein. The optical probe 102 includes one or more walls 103 such as a cylindrical wall or polygonal walls defining an elongate body having a lumen therein. The one or more walls 103 may be rigid, semi-rigid, or flexible. In some examples, the one or more walls 103 may be rigid such as in a probe that is configured as a needle or wand. In some examples, the one or more walls 103 include longitudinally arranged segments that are hingedly connected to each other, such as to provide a steerable, flexible probe. The configuration of the probe may be varied based on the location within the body where the probe is to be used, e.g., for prostrate applications the probe may be more rigid as compared to use in the lung, where the probe may be flexible. In short, the housing for the optical fibers may be optimized for the anatomical location.

The one or more walls 103 may terminate at a distal tip of the optical probe 102. The distal tip may form a flat surface. The distal tip may be substantially perpendicular to the one or more walls 103 thereby. In some examples, the distal tip is oblique with respect to the one or more walls 103, such as forming an angle with respect to the axial length of the one or more walls 103. The distal tip may form a substantially round (e.g., domed or curved surface). The one or more walls 103 and distal tip may be constructed of medical grade, tissue safe polymer or metal.

The space or cavity in the optical probe 102 between the one or more walls 103 may be at least partially filled with material or components, e.g., the cavity may define a passage for optical fibers. For example, the one or more walls 103 may form a solid cylinder of material with apertures therein for the optical fibers 104. Such apertures may be formed by over molding the optical fibers or filler material having the size and shape of the optical fibers.

The optical probe 102 may include a handle or grip spaced from the distal tip. The handle or grip may be sized and shaped to allow health care providers to securely hold and maneuver the optical probe 102.

The one or more walls 103 or distal tip may include apertures therein sized, located, and shaped to allow the optical fibers to fit therethrough. The location of the apertures may be selected based on the desired probe design, such as having some at the distal tip or some around the one or more walls.

The plurality of optical fibers 104 including at least one transmitting fiber 106 and at least one receiving fiber 108. The plurality of optical fibers 104 are disposed within the one or more walls, such as through lumens, apertures, or cavities within the one or more walls 103. The at least one transmitting fiber 106 and at least one receiving fiber 108 are optically transmissive fibers (e.g., fiber optic cables) or other components that can receive and direct light or radiation between different locations. For example, the transmitting fibers 106 are composed to receive light from the at least one light source 110 and direct the light to a location within the body (as determined by the probe 102), and the receiving fibers 108 are composed to receive light fluoresced or reflected from within the body and return the light to the processing elements 120. For example, the receiving fibers 108 may collect diffuse reflectance and/or fluoresced emissions from tissue (e.g., endogenous and/or exogenous fluorophores thereon) under excitation generated by the transmitting fibers 106. Although the transmitting fibers 106 and receiving fibers 108 may include groups or sensing segments (e.g., more than one, fibers, in other embodiments, depending on the tissue to be analyzed, optical transmissivity, and the like) there may be a single fiber for the transmitting fibers 106 and/or receiving fibers 108. Similarly, although the transmitting fibers 106 and receiving fibers 108 have been discussed as separate fibers, in other embodiments, the same fibers that transmit light may receive light as well. The fibers 106 and 108 are positioned along one or more sections or lengths of the probe 102 between the terminal ends (e.g., distal and proximal ends) of the probe 102. Examples of the positioning of the fibers 106 and 108 is discussed in more detail below.

The optical fibers 104 may include one or more optical fibers configured to transmit or receive NIR light, one or more optical fibers formulated to transmit or receive visible light, one or more fibers formulated to transmit or receive ultraviolet light, or combinations of any of the foregoing. For example, the optical fibers 104 may include transmitting fibers for transmitting NIR light, receiving fibers for receiving fluoresced near infrared light, transmitting fibers for transmitting visible light, and receiving fibers for receiving fluoresced visible light. Such examples are useful for optically scanning exogenous and endogenous fluorophores The optical fibers 104 may include a cladding or jacket disposed therearound to prevent loss or contamination of light transmittance. In some examples, the optical fibers 104 include a Super Eska SH4002 multimode plastic fiber, which has a 1.0 mm core diameter made of polymethylmethacrylate (PMMA) with a 1.0 mm cladding made of a fluorinated polymer. The numerical aperture (NA) of the fiber is 0.5 and the loss of the fiber is 190 dB/KM. The fiber is protected by a black polyethylene jacket giving the fiber a total of 4.0 mm diameter. At least one transmitting fiber 106 and at least one receiving fiber 108 may have identical construction. At least one transmitting fiber 106 and at least one receiving fiber 108 may differ in one or more aspects, such as dimension, materials, or cladding.

At least one transmitting fiber 106 and the at least one receiving fiber 108 are operably coupled (e.g., electronically or optically connected) to further elements of the system directly or indirectly via a conduit, cord, or the like.

The form of the optical probe 102 may differ depending upon the desired implementation. For example, the optical probe may be configured as an optical scanning probe sized and shaped to be inserted into a surgical opening or orifice of an animal. The optical probe 102 may be configured as a needle sized and shaped to penetrate into tissue of an animal. Suitable designs for probes are disclosed in U.S. Pat. No. 8,406,858 filed on 1 May 2006, entitled “Multi-Excitation Diagnostic System and Methods for Classification of Tissue,” the disclosure of which is incorporated herein in its entirety by this reference for all purposes.

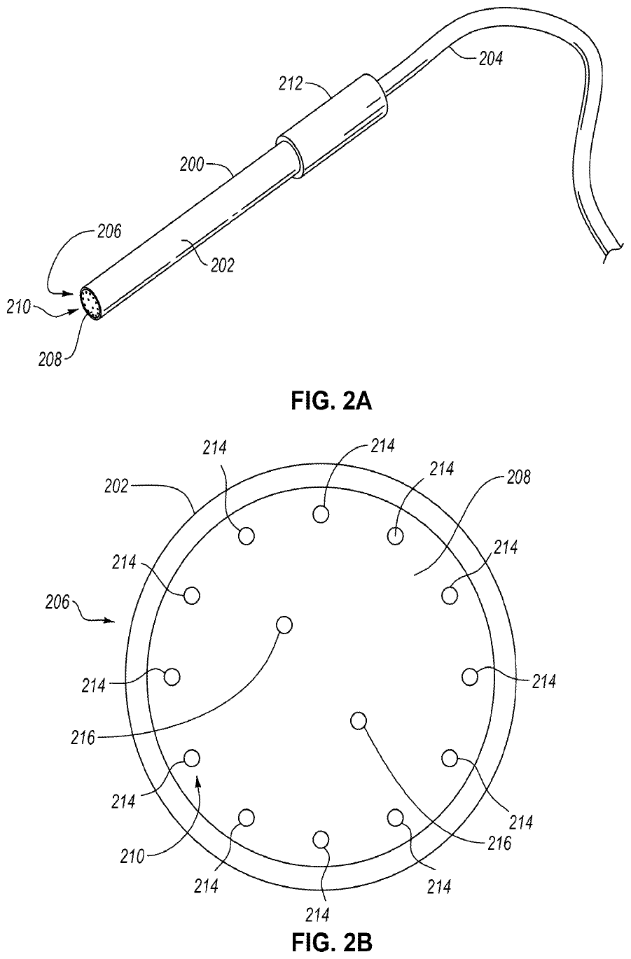

FIG. 2A is an isometric view of an in vivo optical probe 200, according to an embodiment. FIG. 2B is a left end elevation view of the optical probe 200 of FIG. 2A, according to an embodiment. The systems disclosed herein are useful for in situ, real-time diagnosis and identification of abnormal cells such as prostatic carcinomas or lung cancer. The optical scanning probe 200 as shown in FIGS. 2A and 2B may be used to investigate tissue, such as, but not limited to, solid tumors, prostate tissue, and the like. As shown, the probe 200 is primarily composed of a tubular shaft 202 formed by one or more walls terminating distally with a distal tip 206. The tubular shaft 202 is preferably 1 cm or less in diameter. However, the diameter of the probe 200 may vary depending on choice of material, and the wall thickness necessary to withstand pressures and stress levels within the animal body (e.g., rectal cavity, chest cavity, lung tissue).

The tubular shaft 202 houses a plurality of optical fibers 210 that protrude through an outer end wall 208 of the tubular shaft 202 at the distal tip 206 such that the distal ends of the optical fibers 210 are flush with the end wall 208 of the tubular shaft 202. The optical fibers 210 extend proximally through the tubular shaft 202 (e.g., sidewall(s)) to a proximal cap 212, which joins a cable 204 to the tubular shaft 202. The cable 204 provides sheathed protection to the optical fibers 210 as they extend proximally from the probe 200 to connect the probe 200 to attached analytical processing equipment, for example, a spectrometer or other light sensor.

The optical fibers 210 include transmitting fibers 106 and receiving fibers 108 as disclosed herein with respect to FIG. 1. In one implementation, the probe 200 design comprises fourteen fibers as shown in FIGS. 2A and 2B, which include twelve transmitting fibers 214 distributed about the perimeter of the end wall 208 and two receiving (e.g., detector) fibers 216 positioned adjacent the center of the end wall 208. Arrangement of the fibers 210 may be varied and numbers of each of the transmitting fibers 214 and receiving fibers 216 can be greater or lesser, depending on the characteristics of the fibers and the spectra to be transmitted and captured by the fibers. At least some of the transmitting fibers 216 can be coupled to a NIR light source and other transmitting fibers 216 can be coupled to a visible light source. Alternatively, the probe may include only NIR optically coupled fibers 216. The output of a light source(s) may be routed to the probe 200 and controlled by the control circuitry of a computing system operably coupled therewith. The transmitting fibers 214 may thus be illuminated collectively or individually, in sequence or in groupings, and the receiving fibers 216 may be read. The receiving fibers 216 may be coupled to a light sensor and detector or to multiple light sensors and multiple detectors if more than one type of light is emitted (e.g., if both endogenous and exogenous fluorophores are being scanned). For example, receiving fibers 216 composed to receive visible light may be coupled to a first light sensor and first detector while receiving fibers 216 composed to receive NIR light may be coupled to a second light sensor and second detector. Further, it should be noted that while the fibers are discussed as being separately arranged for receiving and transmitting, in other configurations the same fibers that transmit the light may be configured to receive the light reflections from the tissue.

An optical scanning procedure for the diagnosis of abnormal cells and periodic follow-up evaluation may be performed using the probe 200 of FIGS. 2A and 2B. Such procedures may be useful for detecting cancer, such as lung, prostate, or other types of cancer and disease for example, and for monitoring the tissue periodically. This information can be utilized in several different ways to manage the disease: 1) to guide biopsy tools to morphologically adverse locations; 2) to provide comprehensive information during surgery regarding tumor margins, particularly capsule, perforation, and local metastasis; 3) to deliver therapeutic agents directly to cancer foci; and 4) to monitor the response to and success of various therapeutic modalities. Some specific clinical applications include prostate screening, lung screening, brachytherapy, cryotherapy, detection of residual and recurrent disease.

Sufficient depth of light penetration (2-4 cm) and a wide field of view for the probe 200 are obtained by selecting source fibers with small numerical apertures (NA<0.2) and receiving fibers with high numerical apertures (NA>0.5). Intensity modulation of the sources and homodyne demodulation at the output of a fast spectrometer may be used to estimate phase and amplitude variations. The phase and amplitude modulations are then used to determine both the scattering coefficient and the location of a lesion responsible for a scattering event.

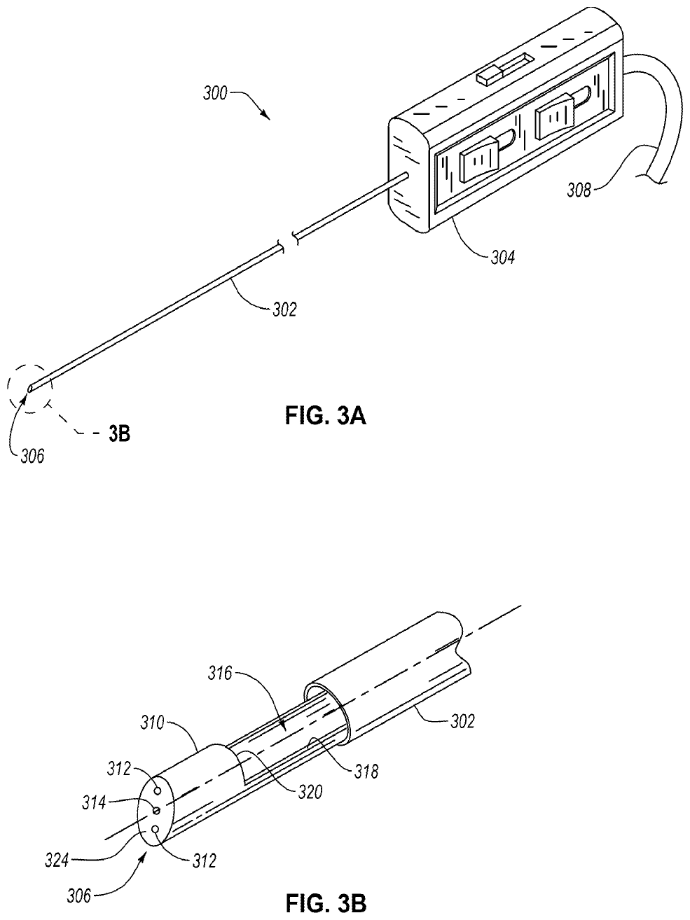

Referring back to FIG. 1, the optical probe 102 may be configured as an optical biopsy and probe device. FIGS. 3A-3B are isometric views of a minimally invasive automatic optical probe may include a biopsy element, such as a needle, brush element, cutter, or the like. The optical probe 300 may be used in the diagnosis of abnormal cells, such as lung cancer, prostate cancer, or the like. The optical probe 300 incorporates an integrated optical probe and may be arranged in a needle configuration, such as for use within the prostrate. The invasive optical probe 300 may be designed to capture elastic scattering spectra (“ESS”) and fluorescence spectra of selected tissues such as lung or prostatic tissues in the range of 200-2,500 nm or even more specifically 700-1,500 nm or 650 nm to 1350 nm.

In one example, the optical probe 300 may be configured as a needle biopsy tool and may include a sheathed needed. For example, the optical probe 300 may include a long outer sheath 302 attached to an actuator handle 304. The outer sheath 302 houses an inner biopsy element 310, such as an inner needle, actuated by the actuator handle 304. A cord sheath 308 extends proximally from the actuator handle 304 to couple the optical probe 300 to analytical equipment as further described below.

The distal tip 306 of the biopsy element, e.g., an inner needle 310 forms and angled blade 324 to assist in the ability of the inner needle 310 to puncture or otherwise be configured to extract cells from tissue. Initially, the inner needle 310 is substantially housed within the outer sheath 302 with only the angled blade 324 extending beyond the distal end of the outer sheath 302. The actuator handle 304 causes the inner needle 310 to extend distally from the distal end of the outer sheath 302 to collect a tissue sample 322 in the standard manner of biopsy needles. The inner needle 310 defines a recessed tray area 316 defined by low sidewalls 318 spaced proximally from a distal tip 306 of the inner needle 310. The inner needle 310 also defines a barb 320 at the top surface of the inner needle 310 at the distal end of the recessed tray area 316 extending proximally toward the recessed tray area 316. The barb 320 assists in extracting and securing a tissue sample within the recessed tray area 316. A typical tissue core sample may be approximately 15 mm long for each biopsy for certain organs. In other examples, such as with lung biopsies, the tissue core sample may be much smaller (e.g., 1-2 mm). In other examples, however, the optical probe 300 may be differently configured, and may include other types of tissue extraction tools, such as, brush or scraping biopsy tools, cutters, or the like. In these configurations, the needle may be omitted and other types of extraction elements may be used.

In embodiments including a needle, the inner needle 310 may be engineered to bring a plurality of optical fibers 312, 314 from the actuator handle 304 to the distal tip 306 flush with the blade 324 surface. However, in other implementations, the fibers may be housed within a housing or sheath separate from the biopsy element. The optical fibers 312, 314 include transmitting fibers 314 and receiving fibers 312. The transmitting fibers 314 and receiving fibers 312 may be similar or identical to the transmitting fibers 106 and receiving fibers 108 (FIG. 1). In one exemplary embodiment, a 16-gauge outer sheath 302 may house a 20-gauge inner needle 310. Three 100-200 μm fibers, one transmitting fiber 314 and two receiving fibers 312 may be threaded through conduits formed within the inner needle 310. Other embodiments may incorporate needles and sheaths of greater or lesser gauge or alternate numbers and diameters of fibers. Excitation of tissue is preferably restricted to the sample point. A small numerical aperture (circa 0.2 or less) may be used for the transmitting fiber 314 to ensure that the excitation does not spread too much over the propagation distance. Larger numerical apertures are preferably used for the receiving fibers 312 in order to maximize the pick-up. Further configurations for the optical fibers 312 and 314 may be utilized.

In actual practice, the optical needle 300 may be inserted into animal tissue, such as the lung or prostate gland, at the desired location and signal normalization performed before activating the light source. The distal tip 306 of the inner needle 310 is positioned inside the tissue. The receiving fiber(s) 312 collect the diffuse reflectance and fluorescence emissions from the tissue under excitation generated by the transmitting fiber(s) 314. Fluorescence and reflectance measurements are then taken to determine whether the adjacent tissue is benign or malignant. Tissue sample collection may thus be limited to areas of tissue that appear to be abnormal based upon spectral analysis. This procedure is repeated and fluorescence and reflectance measurements may be taken from several locations of the tissue.

Optical probes with physical biopsy capabilities may have other forms than the needle probe design disclosed with respect to FIG. 3, such as more robust optical probes (FIGS. 2A-2B) or even steerable probes. To that end, the probes may include flexible housing to allow navigation into certain anatomical areas and relatedly the extraction element may be configured based on the tissue and location to be probed.

Returning to FIG. 1, the optical fibers 104 may be disposed in a steerable or guidable probe as disclosed in U.S. Provisional Patent Application No. 63/185,264 filed on 6 May 2021, entitled “Cancer Diagnostic Device,” the disclosure of which is incorporated herein in its entirety by this reference. For example, transmitting fibers 106 and receiving fiber(s) 108 may be disposed annularly around the at least one wall 103 of a distal end region of a steerable probe. In such examples, the optical fibers 104 may be disposed around at least a portion of a circumference of the probe. The optical fibers 104 face radially outward form the longitudinal axis of the probe. In such example, the optical fibers 104 may be disposed in sensing segments of sequential annular rings of a steerable probe. Such steerable probes may be used in laparoscopes, bronchoscopes, or the like. Such steerable probes may be used to optically examine lung tissue, bronchial tissue, prostate tissue, colon tissue, or the like for abnormal cells or biomarkers of the same using exogenous fluorophores as disclosed herein.

The system 100 may include one or more location or position sensors, such as an accelerometer, gyroscope, positioning stages, multi-axis position sensors, magnetic position sensors, linear motion sensors, rotary position sensor, angle of deflection sensor, encoders, or the like. The position sensors may be located on the probe 102 to provide location data on the position (e.g., location and orientation) of the probe 102 when light signals are detected by the receiving fibers 108. Such data may be used to identify the position of tissue that is irradiated with light from the transmitting fibers (e.g., to ensure the proper location before emitting light) or to note the position of the fluoresced light that is detected by the receiving fibers 108. Accordingly, the location of the tissue may be correlated to the fluoresced light signals to determine the location of the tissue tested. Further, electronic models may be made using the location data and the fluoresced light to build an image of the tissue, such as to show where healthy, questionable, or abnormal (e.g., cancerous) tissue is present in an animal such as a human. Suitable techniques for forming an image or mapping the tissue based at least partially on detected light from tissue are disclosed in U.S. Pat. No. 9,814,449 filed on 6 Jul. 2016, entitled “Motorized Optical Imaging of Prostate Cancer,” the disclosure of which is incorporated herein in its entirety by this reference.

In some examples, the optical probe may include a biopsy tool to take a physical biopsy of the tissue as discussed above with respect to FIG. 3. Such biopsy tools include those disclosed in U.S. Provisional Patent Application No. 63/185,264 filed on 6 May 2021, entitled “Cancer Diagnostic Device,” which has been incorporated herein by reference. For example, the optical probe may include a longitudinal channel therein for a biopsy tool to pass therethrough. The biopsy tool may be selectively advanced, operated, and retracted to take tissue biopsy samples for later analysis. The location of the biopsy may be recorded to create a map of the locations of the biopsies and resultant diagnosis of the tissue(s).

The at least one transmitting fiber 106 is operably coupled to at least one light source 110. The at least one light source 110 is composed to emit light in the near infrared spectrum. For example, the light source may include one or more light generating elements that emit light having a wavelength of at least 750 nm, such as 750 nm to 2500 nm, 780 nm to 1500 nm, 780 nm to 1000 nm, 1000 nm to 1200 nm, less than 1500 nm, or less than 1200 nm. Light in the NIR spectrum may penetrate deeper into tissue than visible or ultraviolet light. For example, light in the NIR range can travel a centimeter into tissue. Accordingly, the devices, systems, and methods disclosed herein may be utilized to scan past the surface of tissue to provide an insight into the health of tissue below the surface.

At least one light source 110 may include one or an array of sources, which may be substantially monochromatic sources or may be broader-band sources. Suitable light sources 110 include inorganic LEDs, organic LEDs, laser diodes, gas lasers, solid-state lasers, fiber lasers, bulbs (halogen, tungsten, xenon, etc.), flash lamps, and the like. Different individual light sources from the array of sources 110 may have (e.g., emit) different wavelengths or wavelength bands. Light sources 110 may generate different wavelengths as desired. For example, a first light source 110 may emit NIR light and a second light source may emit visible light and/or UV light. In embodiments with multiple sources, the light sources 110 may be combined spatially in a multiplexing system, free space, in the fibers, or guided wave, and may be configured such that the light or radiation is delivered via evanescent near-field waves or fair field. In some examples, the light source 110 may emit more than one wavelength of light or more than one range of wavelengths. For example, a light source may emit NIR and one or more of visible and UV light, such as simultaneously or at different, controlled times. Such light sources of multiple wavelengths, such as visible, UV, and NIR are useful to irradiate both exogenous and endogenous fluorophores.

The at least one light source 110 may be equipped to emit light in the visible spectrum and UV spectrum. For example, the light source 110 may include one or more light generating elements that emit light having a wavelength of 380 nm to 700 nm, 380 nm to 500 nm, 500 nm to 700 nm, less than 700 nm, or less than 600 nm. The at least one light source 110 equipped to emit light in the visible spectrum may be the light source or in addition to an NIR light source. Light in the visible spectrum may interact with endogenous fluorophores in tissue more than NIR light. Accordingly, the devices, systems, and methods disclosed herein may be utilized to scan endogenous fluorophores in tissue to provide an insight into the health of tissue. By utilizing both NIR and visible light, exogenous and endogenous fluorophores can be separately examined to determine the health of tissue. For example, endogenous fluorophores may be optically probed to determine the location of healthy tissue and exogenous fluorophores may be optically probed to determine the location of abnormal or suspicious tissue.

The light sources 110 may be operated or otherwise equipped to emit light via different transmitting fibers 106 at different times, such as to receive fluoresced light at different spatial locations at selected times. The light source may be operated or otherwise equipped to emit light from different fibers at the same time. Operation of the at least one light source 110 may be controlled by processing elements 120, such as by a computer 126 therein.

At least one receiving fiber 108 is operably coupled to the one or more processing elements 120. The one or more processing elements 120 are equipped to analyze light received by at least one receiving fiber (e.g., light that is fluoresced from the exogenous fluorophore in the tissue) to determine the health of the tissue. For example, the one or more processing elements are arranged and equipped to determine a presence of abnormal cells in a tissue based on the light fluoresced (e.g., NIR light) from the exogenous fluorophores in the tissue. The processing elements 120 may include at least one light sensor 122, at least one detector 124, and a computer 126.

At least one light sensor 122 is coupled with the optical probe 102 via at least one receiving fiber 108. The at least one light sensor 122 is equipped to translate fluoresced light signals received by the at least one receiving fiber 108 into optical spectral data or signals. The optical spectral data may include wavelength, intensity, timing, duration, modulation of any of the foregoing for the light received and transmitted through the at least one receiving fiber 108. Suitable light sensors 122 may include a spectrometer. For example, a spectrometer can convert light into an electrical signal where each pixel indicates amplitude/intensity and wavelength of the light signal. For example, the light sensor 122 may be configured to receive optical signals from receiving fibers 108 and convert the received light or information into a digital signal. Suitable spectrometers may include a Perkin Elmer absorption spectrometer, an Ocean Optics S2000 double spectrometer with CCD detectors. In one example, a single spectrometer maybe utilized to detect multiple wavelengths of light, such as both visible and NIR light up to about 1000 nm in wavelength. If the wavelength of the NIR light collected is expected to exceed 1000 nm in wavelength, a separate spectrometer equipped to detect NIR light from 750 nm to 1500 nm or 2500 nm may be utilized and another spectrometer equipped to detect visible light may be utilized in parallel so both NIR and visible light are collected. In examples where more than one range of light is collected, such as both visible and NIR light, a first light sensor 122 (e.g., spectrometer) may be used for visible light detection and a second light sensor (e.g., spectrometer) may be used for NIR light detection. Such separate light sensors may be coupled to all receiving fibers or to separate receiving fibers corresponding to the light type expected to be collected by the respective light detectors. A single receiving fiber may be split to two light sensors, such as a first spectrometer equipped to detect NIR light and second spectrometer equipped to detect visible light. Separate receiving fibers may be connected to separate spectrometers, such as a first receiving fiber coupled to a first spectrometer equipped to detect NIR light and second receiving fiber coupled to a second spectrometer equipped to detect visible light.

The light sensors 122 or light detectors may be any type of light capturing or sensing device, including charged-coupled devices, image sensors, or the like. The light sensors 122 may act as an electromagnetic receiver. The light sensors 122 or other components of the system 100 (e.g., processing elements) may then discriminate the received light or electromagnetic radio into n wavelength intervals of the electromagnetic spectrum. For example, the output of the receiver for a single source input may be n (integrated) intensity values, the values representation an integral of intensity values over some interval of the wavelength spectrum. The discrimination of the electromagnetic radiation into n integrated intensity values may be accomplished in a number of manners, including, but not limited to, photodetectors with absorptive filters, spectrometers, and wavelength selected organic photodetectors. Examples of analyzing the received light are discussed in more detail below.

The light sensors 122 (e.g., spectrometers) can determine optical spectral data for a plurality of receiving fibers 108, such as through a plurality of channels therein. In instances where multiple light sources 110 are used, responses from the tissue (e.g., scattering and reflectance of emitted light) may be correlated to individual light sources through time-division multiplexing, space-division multiplexing, or the like. For example, a first light source may be activated and the tissue response captured and then a second light source may be activated and the response captured, etc. Such examples are useful for detecting both exogenous and endogenous fluorophores, such as by irradiating the tissue with light in the NIR and visible light ranges, respectively.

The light sensors 122 may be operably coupled to a detector 124 to translate optical spectral data into digital data. For example, the detector 124 may be electronically coupled to a spectrometer to translate the optical data supplied by the spectrometer into digital data. The detector 124 may comprise a locked detection system. For example, the detector 124, or each of an array of detectors 124 (e.g., each corresponding to a separate receiving fiber), may comprise charge-coupled devices (“CCDs”) or other suitable photodetectors. The detector 124 may be a computational device programed and equipped to translate an optical spectral data (from a spectrometer) to digital data suitable for use in a computer.

The detector 124 is operably coupled (e.g., electronically connected) to the computer 126. The computer 126 is programmed to control the light source 110, receive digital data describing the optical spectral data from the fluoresced light received at the receiving fibers 108, and analyze the digital data to determine the health of the tissue from where the light fluoresced. For example, the computer 126 may include programing stored therein to analyze the spectral character of the fluoresced light to determine if the tissue is malignant, benign, or somewhere in between.

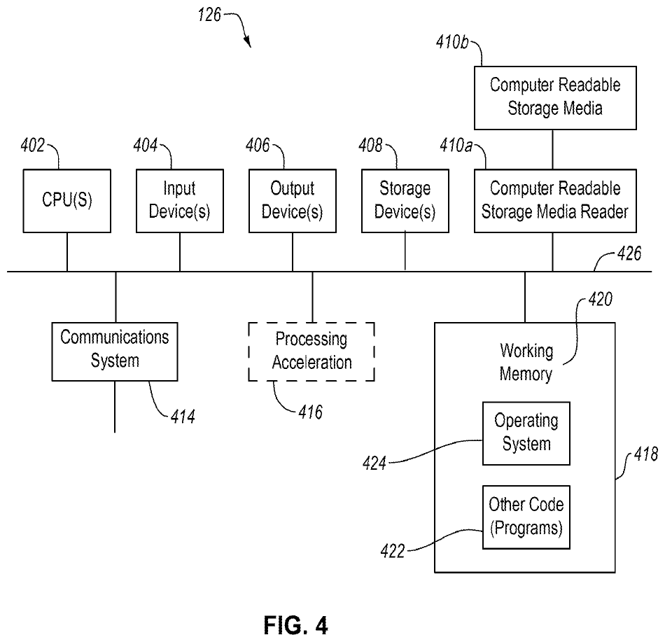

FIG. 4 is a block diagram of the computer 126, according to an embodiment. The computer 126 is shown comprised of hardware elements that are electrically coupled via bus 426. The hardware elements include a processor 402, an input device 404, an output device 406, a storage device 408, a computer readable storage media reader 410a, a communications system 414, a processing acceleration unit 416 such as a DSP or special-purpose processor, and a memory 418. The computer readable storage media reader 410a is further connected to a computer-readable storage medium 410b, the combination comprehensively representing remote, local, fixed, and/or removable storage devices plus storage media for temporarily and/or more permanently containing computer-readable information. The communications system 414 may comprise a wired, wireless, and/or other type of interfacing connection and permits data to be exchanged with external devices.

The computer 126 also comprises software elements, shown as being currently located within memory 420, including an operating system 424 and other code 422, such as a program designed to implement one or more portions of the methods disclosed herein. Variations in the computer 126 may be used in accordance with specific requirements. For example, customized hardware might also be used and/or particular elements might be implemented in hardware, software (including portable software, such as applets), or both. Further, connection to other computing devices such as network input/output devices may be employed.

The memory 420 of the computer 126 includes electronic instructions stored thereon, and executable by the processor 402, for identifying biomarkers indicative of abnormal cells in the tissue of an animal (e.g., human). For example, the computer 126 may include a program for identifying the presence of suspicious (e.g., cancer) cells based on characteristics of optical light data corresponding to fluoresced light emitted from exogenous fluorophores in the tissue responsive to NIR light directed thereto. Fluoresced light from endogenous fluorophores may be collected and analyzed as well. For example, fluoresced light from both exogenous fluorophores and endogenous fluorophores may provide more accurate detection of abnormal cells and edge/margin detection, than only fluoresced light from exogenous fluorophores. In such cases, the different fluorophores may be excited by light from different light sources.

Referring back to FIG. 1, the system 100 analyzes received light to identify healthy cells, abnormal cells, or biomarkers of the same in the tissue. The biomarkers may be indicative of healthy cells or abnormal cells. For example, variations in wavelength, direction, slope, absorption, and the like, can be used to determine whether the tissue is healthy, abnormal, cancerous, or the like. Additionally, margins or boundaries between normal and abnormal or abnormal cells can be identified. Examples of analysis that may be utilized are described in U.S. Pat. No. 8,406,858 filed on 1 May 2006, entitled Multi-Excitation Diagnostic System and Methods for Classification of Tissue,” and U.S. Pat. No. 9,814,449 filed on 6 Jul. 2016, entitled “Motorized Optical Imaging of Prostate Cancer,” which are incorporated by reference herein. Examples include methods including cluster analysis, support vector machines, random forest classification, as well as neural network, deep learning networks and other artificial intelligence (AI) analyses. Specific examples are discussed in more detail below.

Optical spectroscopy provides a methodology to diagnose disease by quantitatively evaluating changes in tissue morphology and composition. Light interacts with exogenous fluorophores (and endogenous fluorophores) attached to biological tissue in a variety of ways. Various types of exogenous fluorophores fluoresce, absorb, and scatter light in different regions of the electromagnetic (EM) spectrum and by different amounts. The optical properties of exogenous and endogenous fluorophores on the tissues are determined by their molecular composition and dimensions. Optical techniques utilize accurate measurements of these absorbed or scattered signals on tissue to identify benign versus malignant tissues. Based on this principle, optical diagnostic techniques can be used to identify various types of precancerous lesions and carcinomas in real-time.

Optical properties of exogenous and endogenous fluorophores attached to different tissues may be very different. Further, optical properties of exogenous fluorophores may differ greatly from naturally occurring endogenous fluorophores in the tissue. For example, exogenous fluorophores may absorb and/or emit more strongly in the NIR spectrum than naturally occurring endogenous fluorophores which absorb and emit more strongly in the visible spectrum. Accordingly, by preferentially attaching exogenous fluorophores to abnormal (e.g., cancerous) cells or biomarkers of the same in tissue (compared to healthy or normal tissue), one may identify the presence, suspicion, or absence of abnormal cells or biomarkers of the same in the tissue based on the presence or absence of characteristic fluorescent spectral data of the exogenous fluorophores corresponding to the NIR spectrum. While the exogenous fluorophores are the focus of the methods and systems herein, endogenous fluorophores may respond to NIR radiation as well. Alternatively, endogenous fluorophores may respond to additional radiation outside of the NIR spectrum. Optical fluorescence data from endogenous fluorophores present in tissues may provide additional information useful for determining the health state of selected tissue. Optical spectroscopic features may need to be established for individual tissue types, endogenous fluorophores, and/or exogenous fluorophores to provide identification of benign versus premalignant/malignant tissue, healthy tissue versus abnormal tissue, or even treated areas of tissue (e.g., necrotic tissue such as by attaching exogenous fluorophores that preferentially bind to heated or otherwise treated tissue). A large majority of scattering events in biological tissue are elastic. Elastic scattering primarily probes morphological features and has proven to be sensitive to histopathologic grade of cancer in different organs. In some instances, endogenous and exogenous signatures may be used to detect abnormal tissue and other instances exogenous signatures are used to detect abnormal tissue.

Fluorescence spectra may be combined with elastic scattering spectra (ESS) to extend the classification for benign versus premalignant/malignant tissue. While fluorescence is a weaker process than elastic scattering, it potentially allows for identification of specific exogenous and/or endogenous fluorophores whose concentrations may vary with disease state. Analysis of both types of observed signals is therefore useful. Such analysis may involve estimation of tissue scattering and fluorescence properties, solution of the scattering problem, and prediction of the effect of scattering and absorption on fluorescence signals.

Data analysis may include matrix assessment as disclosed in U.S. Pat. No. 8,406,858 filed on 1 May 2006, entitled Multi-Excitation Diagnostic System and Methods for Classification of Tissue,” which is incorporated by reference herein. However other techniques for analyzing the data collected by the light fibers and emitted spectra to extract reflection information and changes in the reflected signals that correspond to the tissue characteristics may be used.

In one embodiment, such as with biological tissue, a cluster analysis may be used to separate tissue by characteristics, including, for example, normal, abnormal, as well as optically more refined categories of abnormal, e.g., precancerous or cancerous. The classification and analysis may depend on the type of tissue be analyzed, as well as the data received by the probe. Such cluster analysis may group together items using a statistical analysis of a Euclidean distance measure in three-space generated with the fluorescence, scattering, and absorption measures.

Other methods of data analysis may include principal component, artificial neural networks (ANN), and various statistical analyses that may be employed to develop a preliminary classification scheme for in vivo diagnosis of tissue. For example, classification of ESS (elastic scatting spectra) may be performed in a binary classification scheme using an ANN. Other alternatives include the use of logistic regression, Gaussian processes, support vector machine, random forest, boosted tree regression algorithms to improve the classification scheme. Any of the techniques or methods of data analysis for determining if light signals of fluoresced light from endogenous fluorophores and/or exogenous fluorophores disclosed herein may be stored in the memory of the computer 126.

The computer 126 may store one or more readable and executable programs for controlling the emission of light from the light source 110, tracking and correlating location(s) of the optical probe 102 when light is emitted and/or fluoresced light is detected, building models of the tissue from the detected light using the location data, or performing any portions of the methods and techniques disclosed herein.

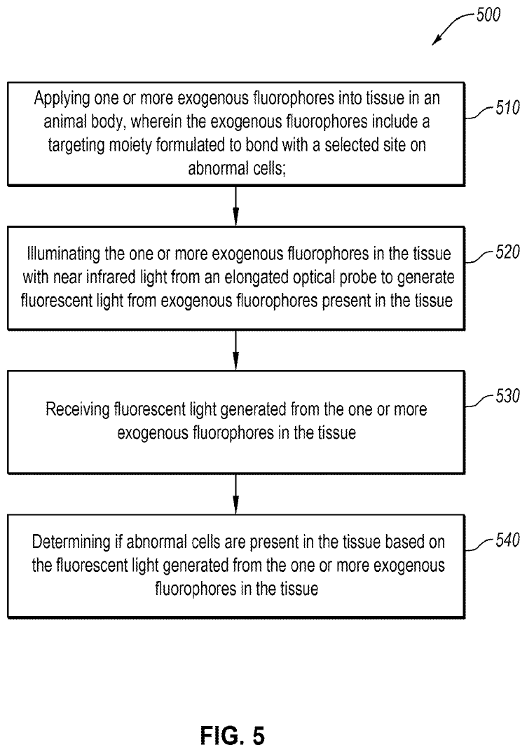

FIG. 5 is a flow diagram of a method 500 for optically determining the presence of abnormal cells in tissue, according to an embodiment. The method 500 includes the block 510 of applying one or more exogenous fluorophores into tissue in an animal body, wherein the exogenous fluorophores include a targeting moiety formulated to bond with a selected site on abnormal cells; the block 520 of illuminating the one or more exogenous fluorophores in the tissue with NIR light from an elongated optical probe to generate fluorescent light from exogenous fluorophores present in the tissue; and the block 530 of receiving fluorescent light generated from the one or more exogenous fluorophores in the tissue; and a block 540 of determining if abnormal cells are present in the tissue based on the fluorescent light generated from the one or more exogenous fluorophores in the tissue. Any of the blocks 510-540 may be divided into multiple blocks, combined with other blocks, or omitted. For example, the block 520 of illuminating the one or more exogenous fluorophores in the tissue with NIR light from an elongated optical probe to generate fluorescent light from exogenous fluorophores present in the tissue may include inserting the probe into the animal and steering the probe to the location of the tissue. Additional blocks may be added to the method 500. For example, the method 500 may include a block of creating an image of the one or more exogenous fluorophores in the tissue based on the fluorescent light generated therefrom.

The block 510 of applying one or more exogenous fluorophores into tissue in an animal body, wherein the exogenous fluorophores include a targeting moiety formulated to bond with a selected site on abnormal cells may include administering the exogenous fluorophores to an animal subject, such as a human in a selected concentration.

Applying one or more exogenous fluorophores may include injecting the exogenous fluorophores to a selected site in the body, such as into or onto a selected tissue. For example, the exogenous fluorophores may be disposed (e.g., injected) directly to a selected region in an animal body, such as an organ, a gland, a suspected tumor site, or the like. Applying one or more exogenous fluorophores into tissue in an animal body may include disposing or injecting the one or more exogenous fluorophores into the animal body to at least a surface of the tissue. Applying the one or more exogenous fluorophores into the animal body to at least a surface of the biological structure includes disposing or injecting the one or more exogenous fluorophores into the tissue to a selected depth therein. The exogenous fluorophores disclosed herein may be utilized below the surface of the tissue because NIR light may penetrate 1 cm into tissue or through tissue 1 cm deep. Accordingly, subsurface detection of cancer may be accomplished using the techniques disclosed herein. Applying one or more exogenous fluorophores into tissue in an animal body includes providing the exogenous fluorophore in intravenous form. The intravenous form may be administered through an IV drip to circulate throughout the animal body. In other implementations, the exogenous fluorophores may be applied by injection into the bloodstream, rather than relying on an IV drip. Other applications may be varied depending on the characteristics of the exogenous fluorophores.

Applying one or more exogenous fluorophores into tissue in an animal body, wherein the exogenous fluorophores include a targeting moiety formulated to bond with a selected site on cancer cells may include applying small molecule organic fluorophores, quantum dot fluorophores, or nanoparticle fluorophores in the tissue. The targeting moieties may be formulated to bind to cancer cells, biomarkers associate with cancer cells, a specific tissue (e.g., organ). For example, the targeting moieties can include functional groups, antibodies, aptamers, or the like. The exogenous fluorophores have an excitation range within the NIR spectrum. The exogenous fluorophores may have an emission range within the NIR spectrum.

Applying one or more exogenous fluorophores into tissue in an animal body includes binding the one or more exogenous fluorophores to the tissue, such as to the tissue or biomarkers associated therewith. The exogenous fluorophores include moieties or structures formulated to bioconjugate to one or more biomolecules in tissue or abnormal cells such (e.g., cancer cells) or biomarkers of the same. Such moieties can be located in coatings or functional groups formulated to bond to specific tissues or structures in animal bodies, such as abnormal cells, or biomarkers of the same, or the like. The exogenous fluorophores may be composed or otherwise formulated to absorb and emit light in a selected spectrum, such as the NIR spectrum. The light emitted from the fluorophores may be detected and the position of the fluorophore may be noted to form an image indicating the presence of the selected tissue (e.g., cancer cells) in the animal body as disclosed herein. Accordingly, the exogenous fluorophores can be used to identify the presence of abnormal cells such as cancer or biomarker of the same in the selected tissue.

### Small Molecule Organic Fluorophores

Small molecule organic fluorophores are among the safest dyes that can be put in the body (e.g., low toxicity). There are a wide range of small molecule organic fluorophores that have excitation and/or emission ranges within the NIR spectrum. The small molecule fluorophores useful for this disclose are water-soluble and carry reactive groups that allow conjugation with antibodies, peptides, or nucleic acids. These dyes can chemically formulated to link one or two biomolecules by controlling the ratio and reaction time. For example, a first amount of fluorophores may be combined with a second amount of antibodies to control the ratio of components based on the first and second amounts, which effects the reaction time of the resulting fluorophore-antibody complex in the tissue. Because of their small molecular weights (e.g., 200-1000 Daltons or 20-100 atoms), when the small molecule fluorophores are chemically linked to larger molecular weight biomolecules, such as antibodies, the pharmacokinetics of the conjugates tend to be similar to those of the biomolecules and not the small molecule organic fluorophores. Accordingly, small molecule organic fluorophores can be administered intravenously without targeting capabilities or can be conjugated with other agents for targeting a specific organ, tumor, or tissue type.

Small molecule fluorophores suitable for use with the devices and methods disclosed herein include cyanine dyes, Alexa dyes, rhodamine dyes, boron-dipyrromethene (“BODIPY”) based NIRF probes, squaraine-based dyes, porphyrin and phthalocyanines derivatives, or the like. The above dyes or indicators can be formulated to fluoresce in the NIR spectrum and to provide excellent NIR fluorescence (NIRF) probes.

Cyanine and Alexa dyes represent classes of dyes that fluoresce with a very high signal to noise ratio. These dyes are non-targeted fluorophores and may be best administered through an IV. Indocyanine green (ICG), for instance, is a tricarbocyanine dye that was permitted by the US Food and Drug Administration (FDA) for photodynamic therapy (PDT) application over 50 years ago. ICG and cyanine derivatives Cy5.5 and Cy7, have been used in imaging for a relatively long time. In animal models, ICG uptake is 1.23 times more in hyperthermia treated tumor than non-hyperthermia treated tumors. Accordingly, ICG can be used to determine if one or more areas of tissue have abnormalities or have been treated. For example, ICG can be used determine efficacy of PDT. After treating a tumor or tissue using PDT (e.g., heat up the tissue above 55 degree Celsius), ICG can be injected and then imaged using the optical probes and techniques disclosed herein to determine efficacy, such as by determining if the margins between healthy and abnormal tissue have moved or if abnormal tissue is still present. For example, heated tissue takes more ICG than non-heated tissue. Accordingly, fluorescence signals from treated tissue (and surrounding healthy tissue) can confirm whether PDT was applied to the tumor, or whether the tumor was partially treated and needs to be retreated for total efficacy.

Some suitable commercial fluorophores may include Alexa dyes, IRDye dyes, VivoTag dyes, and HylitePlus dyes. Some of these newly developed dyes have relatively high brightness due to their high extinction coefficients, and have better characteristics, such as resistance to photobleaching and less non-specific binding. The Alexa dyes are more resistant to photobleaching than the cyanine dyes Cy5.5 and Cy7 dyes. The Cy5.5 dye conjugates produce nonspecific results and rapid liver accumulation. In contrast, Alexa 680 conjugates demonstrated specific targeting with low background providing a high signal-to-noise ratio.

Commercially available heptamethine carbocyanine IR-783 has excellent fluorescence-imaging ability. IR-783 and its derivative MHI-148, have demonstrated potential as optical imaging agents both in vivo and in vitro for the rapid detection of human kidney cancer. Alternatively, two multifunctional NIR fluorescence heptamethine dyes, IR-780 and IR-808, have shown more suitable optical characteristics, good biocompatibility, and with the ability to target against cancer cells. Polyethylene glycol (PEG)ylated IRDye derivative IR-786 shows a large stokes shift, reduced cytotoxicity, and enhanced photostability.

Rhodamine dyes: These dyes belong to the class of xanthene dyes. These dyes may be used as fluorescent probes due to many desirable properties. However, the fluorescence probes designed from classic rhodamine dyes emit visible light only (500-600 nm wavelength) and are thus not suitable for NIR in vivo bioimaging. However, their fluorescence properties can be easily modified and numerous NIR rhodamine derivatives have been developed by modifying the xanthene core, through various mechanisms to regulate fluorescence properties, such as photo-induced electron transfer (PET), oxidation-reduction, and spiro ring opening of xanthenes. For an example, three rhodamine derivatives SiR680, SiR700, and SiR720 have been developed suitable for in vivo fluorescence imaging at wavelength windows of 700 nm and 800 nm. They emitted fluorescence in the NIR region with great potential for biological application. Synthesized via modifying the Si-rhodamine scaffold, SiR680 and SiR700 could also emit strong NIR fluorescence in aqueous media. Another NIR fluorescence rhodamine dye based on tellurium (2-Me TeR) can have selectively probe reactive oxygen species (ROS) utilizing tellurium and could monitor dynamically in vivo endogenous ROS levels. Unique NIR-absorbing xanthene chromophores can be synthesized by modulating the HOMO-LUMO gap in xanthene dyes.

BODIPY-based NIRF probes: BODIPY is the technical common name of a chemical compound with formula C₉H₇BN₂F₂. The common name is an abbreviation for “boron-dipyrromethene.” It is a red crystalline solid, stable at ambient temperature, soluble in methanol. Many BODIPY derivatives may be obtained by replacing one or more hydrogen atoms by other functional groups to form the important class of BODIPY dyes. These organoboron compounds are useful as fluorescent dyes and markers in biological research. BODIPY is advantageous for bioimaging due to high extinction coefficients and quantum yield as well as thermal and photochemical stability. However, absorption and emission wavelengths of classical BODIPY dyes are not in the NIR region. One promising BODIPY derivative, diphenyl dithienyl aza-BODIPY has shown notable red shifts and strong NIR fluorescence. A series of new alpha-bridged linear BODIPY oligomers have displayed strong absorption and high fluorescence efficiency in the NIR region. DC-SPC, a BODIPY derivative, fluoresces in the NIR region and displayed desirable photostability. It has shown superior amphipathy, cell membrane permeability, and specific localization in mitochondria following facile functionalization into DC-SPC-PPh3. Stable NIR BODIPY developed via a novel pyrrole was non-cytotoxic and suitable for bio-imaging assay. In addition to excellent NIR fluorescence emissions, bromo-substituted BODIPY containing thienopyrrole moieties and two novel NIRF BODIPY dyes, each containing two pyridinium groups have shown DNA photocleavage ability through the production of free radicals with potential applications in PDT.

Squaraine-based NIRF probes: Squaraine dyes are a class of organic dyes showing intense fluorescence, typically in the red and NIR region (absorption maxima are found between 630 and 670 nm and their emission maxima are between 650-700 nm). However, the relatively large scale and hydrophobic features of conventional squaraine remain a serious challenge to utilization as an exogenous fluorophore in tissue (e.g., in vitro or in vivo). Squaraine dyes may be used as sensors for ions and, with the advent of protected squaraine derivatives, may be exploited in biomedical imaging using various strategies. For use with the methods and devices herein, the fluorescence signal could be easily turned off or shifted deep into the NIR region. For example, adding dicyanovinyls into the framework of conventional squaraine enhances NIR fluorescence properties and chemical robustness.

Porphyrin and Phthalocyanines derivatives: Porphyrins work well in the context of PDT since they strongly absorb light, which is then converted to energy and heat in the illuminated areas. Phthalocyanines are structurally related to porphyrins. Phthalocyanines and porphyrin derivatives are versatile functional pigments containing four isoindole or pyrrole nitrogen atoms and their emission bands are under the NIR region. However, most of them exhibit absorption and emission maxima below 700 nm. Addition of benzene groups or multiple electron-donating substituents to Phthalocyanines or porphyrin derivatives move the absorption bands from the visible to the NIR spectral region. Conjugated porphyrin dimers with intense absorptions ranging from 650 to 800 nm and fluorescence emission from 700 to 800 nm may be utilized. Hydrophilic porphyrin, such as tetra(hydroxyphenyl)porphyrin (“THPP”) and its derivative (Zn-THPP) may be utilized. Any of the para, meta, and ortho isomers of THPP and ZN-THPP may be utilized. THPP and ZN-THPP rapidly permeate into cells and localize in the nucleus. Accordingly, demonstrating its potential application as a NIR probe for PDT as well as nucleus imaging.

In addition to cyanine dyes, rhodamine derivatives, BODIPYs, porphyrins and squaraines, some other types of NIR fluorophores may be utilized in the small organic molecule fluorophores disclosed herein. Classical dyes, cyanines, and rhodamines have relatively low two photon-absorption cross sections and therefore require high excitation intensity and/or high concentrations when used as fluorochromes in imaging of biological systems. To lower excitation intensity (for reduced damage) or scan more rapidly (dynamic imaging) for detection of species that have very low concentrations, molecules with much larger two-photon absorption brightness than classical dyes may be utilized. For example, octupolar merocyanine chromophores have a much larger two photon-absorption cross section and can be tuned into the NIR region. The colored conjugate base of 1,3-bis(dicyanomethylidene)indan may be utilized an anionic NIRF dye for biomolecule imaging. DC-SPC-PPh3 is a two-photon excitable BODIPY dye that can be used for NIR imaging as well as PDT.

### Quantum Dots (QDs)

QDs may be used as exogenous fluorophores with the devices and methods disclosed herein. Particularly suitable QDs include type II QDs (e.g., QD consists of a Cd/Te core with a Cd/Se or ZnS shell). Specifically, type II QDs that have been encapsulated in polymers having functional groups formulated to conjugate with biomolecules are particularly useful. Other types of QDs may be utilized. The QDs can be formulated to fluoresce in the NIR spectrum and therefore may excellent NIR fluorescence (NIRF) probes.

QDs are semiconductor particles a few nanometers in size, having optical and electronic properties that differ from larger particles due to quantum mechanics. QDs have several advantages in cancer imaging applications over small molecule organic fluorophores including having higher brightness due to their high quantum yield, higher molar-extinction, and higher photo-bleaching thresholds. QDs also have a broad excitation spectra and narrow, symmetric emission spectra that allows QDs to simultaneously detect multiple fluorescent signals using a single excitation light source such as with a CLARI CORE™ optical biopsy system (from Preview Medical, Inc.).

When the quantum dots are illuminated by UV light, an electron in the quantum dot can be excited to a state of higher energy. The excited electron can drop back into the valence band releasing its energy by the emission of light. Their optoelectronic properties change as a function of both size and shape of QDs. Larger QDs of 5-6 nm diameter emits longer wavelengths, with colors such as orange or red. Smaller QDs of 2-3 nm emits shorter wavelengths, yielding colors like blue and green. However, the specific colors vary depending on the exact composition of the QD. Thus, depending on their chemical composition, QDs can emit fluorescence throughout the visible spectrum and into the NIR region.

Type II QDs emit fluorescence in the far-red and NIR range, which makes them ideal for in vivo cancer imaging with the methods and devices disclosed herein. The majority of QDs are binary semiconductor crystals composed of two types of atoms from the II/VI or III/V group elements on the periodic table. However, many of these QDs may not be desirable for in vivo imaging due to their short emission wavelength. For example, Cadmium/Selenium (Cd/Se) QDs has an emission wavelength of 470-655 nm.

In vivo imaging with QDs may be hindered by their potential toxicity, relatively large size, and short circulation time. Water solubility of QDs is important to their biological applications. To achieve water solubility and biocompatibility, QDs are can be encapsulated with various types of amphiphilic polymers, which increase their hydrodynamic radius to about 10-20 nm. For example, 40% amine-modified poly(acrylic acid) (PAA), may be used as an effective amphiphilic polymer to impart water solubility to hydrophobic QDs. These amphiphilic polymers often carry chemically reactive groups, such as amines and carboxylic acids that allow conjugation with biomolecules such as peptides, proteins, and nucleic acids. Encapsulation and bioconjugation do not usually alter the optical properties of QDs significantly. Accordingly, QDs coated with an amphiphilic polymer having functional groups or structural features that allow attachment (e.g., conjugation) to biomolecules are particularly useful as exogenous fluorophores.

### Nanoparticles (NP)

Nanoparticles that have been conjugated with a polymer (e.g., PEG) coating tuned for bioconjugation may be used as exogenous fluorophores with the devices and methods disclosed herein. For example, fluorophore-doped nanoparticles encapsulated by amphiphilic polymers may be particularly useful with the methods and devices disclosed herein.

Advancement in nanotechnology area has enabled fabrication of wide variety of metallic and nonmetallic nanostructures including QDs, nanodots, nanorods, nanotubes, nanoclusters etc. While one of these NPs may not fluoresce by itself as QDs, they can be conjugated with fluorophores described above for biomedical applications in NIR spectrum. Functionalization of NPs can be achieved using polymer coating (ex: PEG) that will also the reduce toxicity of some nanoparticles. For an example, magnetic nanoparticles made of Gadolinium (Gd3+ cations) are highly toxic. However, reversible additional fragmentation chain transfer (“RAFT”) polymerization of Gd NPs prevents exposure of Gd3+ making it possible for in vivo applications. By functionalization with magnetic NPs and polymers, the stokes shift of a fluorophore (e.g., IR-820) could be tuned.

Fluorophore-doped nanoparticles encapsulated by amphiphilic polymers tend to have high brightness in NIR due to the large number of encapsulated fluorophores. Also have high photostability due to their polymer coating, which prevents penetration of oxygen and reduces bleaching. NPs with a core-shell architecture and NIR fluorescent organic dyes embedded non-covalently or covalently show enhanced photostability and biocompatibility, low self-aggregation, bright fluorescence signal, with bioconjugation that is easily tunable. By combining with NPs, indocyanine green (“ICG”) molecules could avoid aggregation and reduce self-quenching. A self-assembled ICG-containing nanostructure using ICG and phospholipid-PEG (PL-PEG) can increase the NIR-dependent temperature efficiently and could target tumor cells when further linked with an antibody. NIR fluorescent albumin NPs using human serum albumin (HSA) and a NIR dye derivative could specifically target colon cancer and even undistinguishable tumors that can only otherwise be confirmed by histopathological analysis. These NPs can demarcate the tumor with high resolution, demonstrating the capability of both optical imaging and photothermal treatment.

Gold nanostructures have many favorable properties ideal for in vivo biomedical applications, such as safety, availability, and compatibility. They can be used for Raman imaging and photoacoustic imaging (PAI) mainly due to their excellent optical properties, high stability, lower toxicity, and bioconjugation simplicity. Fluorescent gold nanoprobes (GNP), characterized by ultra-small size, tunable optical properties, and considerable biocompatibility, may be utilized as targeted probes for in vivo optical imaging. A gold nanorod constructed by the modification on poly-(allylamine hydrochloride) followed by Rose Bengal (RB) molecule conjugation, provides significant NIR absorption and emission, excellent photostability, good biocompatibility, and specific identification of oral cancer cells.

In some examples, the exogenous fluorophores are composed to bond with biomarkers synonymous with abnormal cells, such as cancer cells. The exogenous fluorophores, or moieties bound thereto, may be composed to bond with antibodies, aptamers, or the like which preferentially target (e.g., bond with) biomarkers of abnormal cells. For example, the biomarker may include prostate specific membrane antigen (PSMA) or the like and the exogenous fluorophore may be composed to bond with the biomarker. In some examples, the fluorophores are composed to bond with tissue which may be susceptible to cancer, such as with one or more moieties of the exogenous fluorophores. In such examples, the exogenous fluorophores may fluoresce light differently in healthy cells than in abnormal cells. Accordingly, the method 500 may be utilized to determine the health of tissue regardless of the exogenous fluorophore's ability to bind directly to the abnormal cells.

After the exogenous fluorophores are applied to the tissue. The exogenous fluorophores may bond with the tissue or biomarkers associated therewith for a duration, such as until cleared by natural processes in the body (e.g., circulation of fluids). The half-life time of a specific exogenous fluorophore is the time it takes for half of the exogenous fluorophore to be cleared from the tissue in the body, thereby reducing the concentration of the fluorophore by half in the half-life time. The duration or half-life time may be as short as 15 minutes and as long as an hour, such as 15 minutes to 45 minutes. The excitation and receiving fluoresced light from the exogenous fluorophores may be performed within the half-life time of the one or more fluorophores. For example, in the case of multiple fluorophores, such acts are performed within the half-life time of a shortest lived exogenous fluorophores.

The block 520 of illuminating the one or more exogenous fluorophores in the tissue with NIR light from an elongated optical probe to generate fluorescent light from exogenous fluorophores present in the tissue includes emitting NIR light in any of the NIR wavelengths or range disclosed herein. The block 520 includes using an elongated optical probe that is configured as an optical probe. For example, the elongate optical probe may be configured as any of the optical probes disclosed herein. The optical probe may be a needle sized and shaped to penetrate the tissue of an animal, may be an optical scanning probe, or may be a steerable probe as disclosed herein. Illuminating the one or more exogenous fluorophores in the tissue with NIR light from an elongated optical probe to generate fluorescent light from the one or more exogenous fluorophores present in the tissue includes emitting NIR light into the tissue in vivo.

The block 520 of illuminating the one or more exogenous fluorophores in the tissue with NIR light from an elongated optical probe to generate fluorescent light from exogenous fluorophores present in the tissue includes positioning a distal end of the elongated optical probe in contact with or adjacent to the tissue. Such positioning may include inserting the elongated optical probe into the animal, such as via an orifice of the animal or an incision in the animal. Positioning the distal end of the elongated optical probe in contact with or adjacent to the tissue may include inserting an optical scanning probe into the animal, such as into the rectum of a human male to contact the prostate of the subject. Positioning the distal end of the elongated optical probe in contact with or adjacent to the tissue may include inserting an optical scanning probe into the esophagus of a human to come into contact with the esophagus, bronchi, lungs, or stomach. Positioning the distal end of the elongated optical probe in contact with or adjacent to the tissue may include inserting a optical probe shaped as a needle into a tissue (e.g., organ, gland, or other tissue) through a surgical opening in the animal. For example, the optical probe may be inserted into a lung or other organ through a surgical incision to emit and receive light from exogenous fluorophores bound thereto.

In some examples, the optical probe may be configured as a steerable probe and inserted into a lung, prostate, liver, stomach, bladder, bowel, other organ, breast, or other tissue structure through a surgical incision, orifice, or both to emit light and receive light from exogenous fluorophores bound thereto. The steering may be performed by a computer or healthcare professional using a handle of the probe. The optical probe may be inserted by being attached to a guide wire and threaded into location of the tissue by the guide wire. In some examples, the optical probe may be inserted on its own with the structure of the probe acting as the guide wire. The insertion may be based on a particular orifice or entry point depending on the tissue to be analyzed, such as via the mouth for entry into the lungs, via the rectum for entry to the colon, etc. The optical probe may be steered or otherwise positioned using an imaging device, such as ultrasound imaging, x-ray, or the like.

The optical probe may be steered to a position at a selected tissue such as via the handle, computer, and imaging to test abnormal tissue or tissue of interest.

Illuminating the one or more exogenous fluorophores in the tissue with NIR light from an elongated optical probe to generate fluorescent light from one or more exogenous fluorophores present in the tissue and receiving fluorescent light generated from the one or more exogenous fluorophores in the tissue are performed within the half-life time of the shortest lived exogenous fluorophore of one or more exogenous fluorophores introduced into the animal body (e.g., 15 minutes to 45 minutes after applying one or more exogenous fluorophores into tissue in an animal body).

Illuminating the one or more exogenous fluorophores in the tissue with NIR light may be carried out for a selected duration or at specific times when multiple transmitting fibers and multiple receiving fibers are utilized. Illuminating the one or more exogenous fluorophores in the tissue may include illuminating one or more endogenous fluorophores with the infrared light or with a different spectrum of light, such as visible or ultraviolet.

The block 530 of receiving fluorescent light generated from the one or more exogenous fluorophores in the tissue includes receiving the fluorescent light in at least one receiving fiber of any of the optical probes disclosed herein. Receiving fluorescent light generated from the one or more exogenous fluorophores in the tissue includes receiving fluorescent light in the NIR spectrum with any of the optical probes disclosed herein. Receiving fluorescent light generated from the one or more exogenous fluorophores in the tissue includes receiving fluorescent light in a discrete portion of the NIR spectrum such as a specific wavelength or range of wavelengths.

Receiving fluorescent light generated from the one or more exogenous fluorophores in the tissue may include mapping a position of the fluorescent light detected with the optical probe or receiving fibers therein. The location where the fluorescent light is received may be recorded from location sensors and may be recorded with spectral data of the received fluorescent light at the location. A map of characteristics may be constructed from the location data and corresponding received light spectral data. The map may be displayed or output as an image or model of the tissue. The image may display the health of the tissue at different locations after the fluorescent light at the locations is used to determine the health of the tissue thereat. Accordingly, the method 500 may include creating an image of the one or more exogenous fluorophores in the tissue based on the fluorescent light generated therefrom.

Receiving fluorescent light generated from the one or more exogenous fluorophores in the tissue includes receiving the light in the one or more processing elements, such as at a light sensor (e.g., spectrometer) to produce fluorescent spectral data. The fluorescent spectral data may be converted to digital data in the detector 124. The digital data may be communicated to a computer with executable instructions for determining if the digital data shows that the received florescent light indicates the tissue is healthy, abnormal, or questionable (e.g., further testing is needed).

Receiving fluorescent light generated from the one or more exogenous fluorophores in the tissue may include receiving fluorescent light generated from the one or more exogenous fluorophores in the tissue such as responsive to NIR light excitation. By illuminating the tissue with only NIR light, exogenous fluorophores may be preferentially examined because the exogenous fluorophores are specifically formulated to emit light responsive to NIR light excitation. In such examples, light emitted from endogenous fluorophores which typically respond to UV and visible light may be limited.

In some examples, fluoresced light from the one or more endogenous fluorophores may also be received by the optical probe as described herein with respect to receiving the fluoresced light from the exogenous fluorophores. The fluoresced light from endogenous fluorophores may be received at a different time (e.g., sequentially) than the fluoresced light from the exogenous fluorophores, such as responsive to excitation from different light ranges at different times. The light fluoresced from the endogenous fluorophores may be utilized in mapping, imaging, or determining the presence of abnormal cells as disclosed herein, such as in addition to, or alternative to, the fluoresced light from the one or more exogenous fluorophores.

The block 540 of determining if abnormal cells are present in the tissue based on the fluorescent light generated from the one or more exogenous fluorophores in the tissue may include performing any of the techniques disclosed herein for differentiating between the characteristics of fluoresced light from exogenous fluorophores on healthy tissue and cancerous tissue (consider treated tissue and abnormal but benign tissue). The digital data of the characteristics of the fluorescent light (e.g., wavelength, intensity, duration, modulation, etc.) from the exogenous fluorophores or endogenous fluorophores may be utilized to determine if the tissue is healthy, abnormal, treated, or questionable as disclosed herein. For example, determining if suspicious (e.g., cancer) cells are present in the tissue based on the fluorescent light generated from the one or more exogenous fluorophores in the tissue.

Determining if abnormal cells are present in the tissue based on the fluorescent light generated from the one or more exogenous fluorophores may include utilizing fluorescent light generated from the one or more endogenous fluorophores, such as for margin detection of abnormal cells. The light from exogenous and endogenous fluorophores may be collected at different times (e.g., sequentially) responsive to different excitation light ranges, such as NIR light first and then UV light and visible light. The fluorescent light generated from the one or more endogenous fluorophores may be processed using any of the techniques disclosed herein for determining the presence of abnormal cells. The fluorescent light generated from the one or more endogenous fluorophores may be used in addition to or as an alternative to fluoresced light from the exogenous fluorophores.

The method 500 may include examining endogenous fluorophores to identify and locate tissue that is healthy. For example, the method 500 may include illuminating the tissue with visible radiation from the elongated optical probe to generate fluorescent light from one or more endogenous fluorophores present in the tissue, receiving fluorescent light generated from the one or more endogenous fluorophores in the tissue, and identifying if healthy cells are present in the tissue based on the fluorescent light generated from the one or more endogenous fluorophores in the tissue. Endogenous fluorophores present in healthy tissue react differently to UV and visible light than abnormal cells. The healthy tissue may be identified using a matrix assessment of the fluorescent light generated from the one or more endogenous fluorophores. The healthy tissue may be identified based on comparison of data to know healthy tissue, such as by gathering baseline measurements in healthy tissue. In such examples, abnormal tissue may be defined, at least in part, as any tissue that exhibits fluorescence that differs from the healthy tissue.

The endogenous and exogenous fluorophores may be irradiated at the same time or different times. For example, if irradiated at the same time, the respective fluorophores emit light in different ranges of electromagnetic radiation. For example, the exogenous fluorophores emit light in the NIR range and the endogenous fluorophores emit light in the visible range. The respective fluoresced light may be processed separately to determine the presence of healthy and abnormal tissue.

The method 500 may include locating margins between healthy cells and abnormal cells based on position data of the fluorescent light generated from the one or more exogenous fluorophores and one or more endogenous fluorophores in the tissue.

The method 500 may include determining the health of tissue in a longitudinal space within the animal or specific organ thereof Such a longitudinal space may be a tunnel longitudinally spaced along the optical probe. For example, such a determination may be carried out by sequentially emitting light, detecting fluoresced light in a location, and then moving the probe longitudinally through tissue of the subject, and repeating the illumination and detection steps at the new location(s). For example, the method 500 may include building a three dimensional map of relative health of tissue by moving the optical probe to a new location longitudinally spaced from a prior location by a distance and repeating the illuminating, receiving and determining steps with NIR light and repeating the illuminating, receiving, identifying, and locating steps with visible light to determine a location of healthy cells and abnormal cells. The relative location of the detected light may be tracked using location data from the optical probe, recorded, and processed to build the three dimensional map. The three dimensional map may be utilized to guide treatment or determine the efficacy of treatment.

The method may include determining the health of the tissue based on one or more classifiers. Such classifiers may include elastic scattering spectra, fluorescence lifetime, or Raman scattering. These classifiers may be used to increase the sensitivity/specificity for tissue classification, localization (3D mapping), monitor response to therapy, and recurrence of the disease.

In some examples, the method 500 may include treating the identified tissue, such as if the tissue is cancerous. Treatment may include resection, ablation, cryotherapy, medication, (e.g., chemotherapy), or the like. The treatment may be performed using the optical probe or another probe such as a laparoscopic surgical device.

The method 500 may include determining an efficacy of the treatment by differential analysis. For example, by repeating the treatment, illuminating, receiving and determining steps with NIR light and repeating the illuminating, receiving, identifying, and locating steps with visible light, the location of the margins between healthy cells and abnormal cells can be monitored for movement. If the margin moves to show a smaller area of abnormal cells, it can be determined that the treatment is working. If the margin does not move or shows a larger area of abnormal cells, it can be determined that the treatment is not working.

The systems and methods disclosed herein utilized NIR light on optical scanning probes to simulate NIR emission from exogenous (and endogenous fluorophores) on the tissue because there is little interference between exogenous fluorophores and endogenous fluorophores in the NIR spectrum. Accordingly, exogenous fluorophores are formulated to target and preferentially attach to abnormal cells, healthy tissue of interest, or biomarkers of the same to optically determine the health of the tissue using fluorescent light emitted from the fluorophores in response to irradiation by NIR light. By utilizing NIR light and exogenous fluorophores that interact with and emit NIR light, the systems and techniques herein can avoid many of the obstacles presented by interference with fluoresced light from endogenous fluorophores, such as blood interference. By utilizing NIR light, tissue may be examined below the surface of the tissue because NIR light can penetrate tissue up to centimeters. Accordingly, the methods and systems herein can determine the health of tissue on a three-dimensional scale.

As used herein, the term “about” or “substantially” refers to an allowable variance of the term modified by “about” by ±5% or ±1%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

1. A method for optically determining the presence of abnormal cells in tissue, the method comprising:
applying one or more exogenous fluorophores into tissue in an animal body, wherein the exogenous fluorophores include a targeting moiety formulated to bond with a selected site on abnormal cells;
illuminating the one or more exogenous fluorophores in the tissue with near infrared light from an elongated optical probe to generate fluorescent light from exogenous fluorophores present in the tissue;
receiving fluorescent light generated from the one or more exogenous fluorophores in the tissue; and
determining if abnormal cells are present in the tissue based on the fluorescent light generated from the one or more exogenous fluorophores in the tissue.

2. The method of claim 1 wherein applying one or more exogenous fluorophores into tissue in an animal body includes injecting the one or more exogenous fluorophores into the animal body to at least a surface of the tissue.

3. The method of claim 2 wherein injecting the one or more exogenous fluorophores into the animal body to at least a surface of the tissue includes injecting the one or more exogenous fluorophores into the tissue to a selected depth therein.

4. The method of claim 2 wherein injecting the one or more exogenous fluorophores into the animal body to at least a surface of the tissue includes administering the one or more exogenous fluorophores into the animal body intravenously.

5. The method of claim 1 wherein the one or more exogenous fluorophores include at least one of a small molecule organic fluorophore, quantum dots, or nanoparticles composed to fluoresce near infrared light when irradiated by one or more of visible light, ultraviolet light, or near infrared light.

6. The method of claim 1 wherein the one or more exogenous fluorophores include one or more of a cyanine dye, an Alexa dye, a rhodamine dye, a boron-dipyrromethene-based NIRF dye, a squaraine-based dye, or a porphyrin dye, or a phthalocyanines derivative.

7. The method of claim 1 wherein the one or more exogenous fluorophores include type II quantum dots that have been encapsulated in an amphiphilic polymer having functional groups formulated to conjugate with selected biomolecules.

8. The method of claim 1 wherein the one or more exogenous fluorophores include fluorophore-doped gold or gadolinium nanoparticles encapsulated by an amphiphilic polymer having functional groups formulated to conjugate with selected biomolecules.

9. The method of claim 1 wherein applying one or more exogenous fluorophores into tissue in an animal body includes binding the one or more exogenous fluorophores to the tissue.

10. The method of claim 1 wherein illuminating the one or more exogenous fluorophores in the tissue with near infrared light from an elongated optical probe to generate fluorescent light from one or more exogenous fluorophores present in the tissue and receiving fluorescent light generated from the one or more exogenous fluorophores in the tissue are performed are performed within the half-life time of the one or more exogenous fluorophores in an animal body.

11. The method of claim 1 wherein illuminating the one or more exogenous fluorophores in the tissue with near infrared light from an elongated optical probe to generate fluorescent light from the one or more exogenous fluorophores present in the tissue includes emitting near infrared light into the tissue in vivo.

12. The method of claim 1 wherein receiving fluorescent light generated from the one or more exogenous fluorophores in the tissue includes mapping a position of the fluorescent light detected.

13. The method of claim 1, further comprising creating an image of the one or more exogenous fluorophores in the tissue based on the fluorescent light generated therefrom.

14. The method of claim 1 wherein determining if abnormal cells are present in the tissue based on the fluorescent light generated from the one or more exogenous fluorophores in the tissue includes performing a matrix assessment of the fluorescent light generated from the one or more exogenous fluorophores.

15. The method of claim 1, further comprising:
illuminating the tissue with visible radiation from the elongated optical probe to generate fluorescent light from one or more endogenous fluorophores present in the tissue;
receiving fluorescent light generated from the one or more endogenous fluorophores in the tissue; and
identifying if healthy cells are present in the tissue based on the fluorescent light generated from the one or more endogenous fluorophores in the tissue.

16. The method of claim 15, further comprising locating margins between healthy cells and abnormal cells based on position data of the fluorescent light generated from the one or more exogenous fluorophores and one or more endogenous fluorophores in the tissue.

17. The method of claim 16, further comprising:
applying a treatment to the abnormal cells; and
determining an efficacy of the treatment by repeating the applying, illuminating, receiving and determining steps with NIR light and repeating the illuminating, receiving, identifying, and locating steps with visible light to determine if the location of margins between healthy cells and abnormal cells has moved based on the treatment.

18. The method of claim 17 wherein the treatment includes ablation, chemotherapy, irradiation, or tissue resection.

19. The method of claim 15 further comprising building a three dimensional map of relative health of tissue by moving the optical probe to a new location longitudinally spaced from a prior location by a distance and repeating the illuminating, receiving and determining steps with NIR light and repeating the illuminating, receiving, identifying, and locating steps with visible light to determine a location of healthy cells and abnormal cells.

20. An optical biopsy system comprising:
an elongate optical probe with a distal tip;
a plurality of optical fibers comprising at least one transmitting fiber and at least one receiving fiber, wherein the plurality of optical fibers extend within the optical probe and terminate in the distal tip of the optical probe; and
at least one light source operatively coupled to the at least one transmitting fiber and configured to generate light in at least the near infrared spectrum;
one or more processing elements configured to determine a presence of abnormal cells in a tissue based on fluoresced light from one or more exogenous fluorophores composed to fluoresce near infrared light responsive to excitation with near infrared light.

21. (canceled)

22. (canceled)

23. (canceled)

24. (canceled)

25. (canceled)

26. (canceled)

27. (canceled)

28. (canceled)
